(19) United States
(12) Patent Application Publication
(10) Pub. No.: US 2015/0170075 A1
(43) Pub. Date: Jun. 18, 2015

(54) METHOD, APPARATUS, AND COMPUTER PROGRAM PRODUCT FOR CREATING LOGICAL UNITS

(71) Applicant: MCKESSON FINANCIAL HOLDINGS, Hamilton (BM)

(72) Inventors: Bryan Self, Birmingham, AL (US); Josh Turner, HueyTown, AL (US); Preston Prewett, Sylvan Springs, AL (US)

(73) Assignee: MCKESSON FINANCIAL HOLDINGS, Hamilton (BM)

(21) Appl. No.: 14/106,898

(22) Filed: Dec. 16, 2013

(51) Int. Cl. G06Q 10/06; G06Q 50/22

(52) U.S. Cl. CPC G06Q 10/0633; G06Q 50/22

(57) ABSTRACT

A method is provided for creating, configuring, and displaying logical units. A logical unit may include underlying data defining subunits defined based on a physical location and may be stored in a centralized system. Upon request from a client terminal, a graphical logical unit may provide a visual representation of the subunits, or rooms. A user may view specified rooms from various areas in a consolidated view, as opposed to accessing a geospatial arrangement for each individual area of interest.

### TECHNOLOGICAL FIELD

Embodiments of the present invention relate generally to computer technology and, more particularly, to methods, apparatuses, and computer program products for creating logical units and displaying visual representations of logical units.

### BACKGROUND

The widespread use of modern computing technology and improvements in visual media has led to an increasing demand for visual representations of data. The healthcare industry, among others, is one that utilizes geospatial arrangement to convey information in a visual format, efficiently and effectively to various units and individuals, such as throughout a hospital, for example. Doctors and staff may view a floor plan of patient rooms for a particular floor of a hospital to manage workflows and attend to patients. Extensive information relating to a specified location or area of a building may therefore be presented in a single visual representation.

### BRIEF SUMMARY OF SOME EXAMPLE EMBODIMENTS

Methods, apparatuses, and computer program products are therefore provided for creating graphical logical units. According to example embodiments, graphical logical units may be created for a user to create a customized view of information specific to a variety of rooms, floors and/or other locations that otherwise may need to be accessed by viewing a number of different geospatial arrangements. Medical facilities in particular may benefit from configurable logical units so that doctors, managers, or other users overseeing rooms or staff members on a number of different floors, for example, may access the visual information in a consolidated view. Example embodiments also provide for cloning and editing of logical units for customized logical units and sharing amongst users.

A method is provided for creating a logical unit, the method comprising receiving an indication of a selection of a plurality of subunits, wherein each of the selected subunits is defined based on a physical location, with processing circuitry, storing data defining the logical unit, wherein the data identifies each of the selected subunits and uniquely identifies the logical unit, and in response to receiving a request to access the logical unit, causing display of a visual representation of the selected subunits, wherein the selected subunits are positioned without respect to their corresponding geospatial arrangements.

In some embodiments, receiving the indication of the selection of the plurality of subunits further comprises receiving an indication of a selection of a subset of all available subunits, wherein the subset comprises less than all of the available subunits, and wherein at least two of the selected subunits are physically located remotely from one another. In some embodiments, the corresponding geospatial arrangements provide information regarding the subunits' physical location respective to other subunits. The visual representation may convey pertinent information specific to at least one subunit. In some embodiments, the method includes copying the logical unit as a new logical unit, receiving user input to configure the new logical unit, and storing additional data defining the new logical unit.

In some embodiments, each of the subunits comprises a room in a building, and the corresponding geospatial arrangements are defined by one or more floor plans.

The selected subunits may be grouped in the visual representation based on their respective physical locations. The plurality of subunits may be selected based on a characteristic other than the respective physical locations.

A computer program product for creating a logical unit is also provided, the computer program product comprising at least one non-transitory computer-readable medium having computer-readable program instructions stored therein, the computer-readable program instructions comprising instructions, which when performed by an apparatus, are configured to cause the apparatus to at least receive an indication of a selection of a plurality of subunits, wherein each of the selected subunits is defined based on a physical location, store data defining the logical unit, wherein the data identifies each of the selected subunits and uniquely identifies the logical unit, and in response to receiving a request to access the logical unit, cause display of a visual representation of the selected subunits, wherein the selected subunits are positioned without respect to their corresponding geospatial arrangements.

In some embodiments, receiving the indication of the selection of the plurality of subunits further comprises receiving an indication of a selection of a subset of all available subunits, wherein the subset comprises less than all of the available subunits, and wherein at least two of the selected subunits are physically located remotely from one another. In some embodiments, the corresponding geospatial arrangements provide information regarding the subunits' physical location respective to other subunits. The visual representation may convey pertinent information specific to at least one subunit. In some embodiments, the computer program product further includes instructions for copying the logical unit as a new logical unit, receiving user input to configure the new logical unit, and storing additional data defining the new logical unit.

In some embodiments, each of the subunits comprises a room in a building, and the corresponding geospatial arrangements are defined by one or more floor plans.

The selected subunits may be grouped in the visual representation based on their respective physical locations. The plurality of subunits may be selected based on a characteristic other than the respective physical locations.

An apparatus for creating a logical unit, the apparatus comprising processing circuitry configured to communicate with a third party system and client, said processing circuitry configured to cause the apparatus to at least receive an indication of a selection of a plurality of subunits, wherein each of the selected subunits is defined based on a physical location, store data defining the logical unit, wherein the data identifies each of the selected subunits and uniquely identifies the logical unit, and in response to receiving a request to access the logical unit, cause display of a visual representation of the selected subunits, wherein the subunits are positioned without respect to their corresponding geospatial arrangements.

In some embodiments, receiving the indication of the selection of the plurality of subunits further comprises receiving an indication of a selection of a subset of all available subunits, wherein the subset comprises less than all of the available subunits, and wherein at least two of the selected subunits are physically located remotely from one another. In some embodiments, the corresponding geospatial arrangements provide information regarding the subunits' physical location respective to other subunits. The visual representation may convey pertinent information specific to at least one subunit. In some embodiments, the processing circuitry is further configured to cause the apparatus to copy the logical unit as a new logical unit, receive user input to configure the new logical unit, and store additional data defining the new logical unit.

In some embodiments, each of the subunits comprises a room in a building, and the corresponding geospatial arrangements are defined by one or more floor plans.

The selected subunits may be grouped in the visual representation based on their respective physical locations. The plurality of subunits may be selected based on a characteristic other than the respective physical locations.

An apparatus for creating a logical unit is provided, with means for: receiving an indication of a selection of a plurality of subunits, wherein each of the selected subunits is defined based on a physical location, storing data defining the logical unit, wherein the data identifies each of the selected subunits and uniquely identifies the logical unit, and in response to receiving a request to access the logical unit, causing display of a visual representation of the selected subunits, wherein the subunits are positioned without respect to their corresponding geospatial arrangements.

The above summary is provided merely for purposes of summarizing some example embodiments of the invention so as to provide a basic understanding of some aspects of the invention. Accordingly, it will be appreciated that the above described example embodiments are merely examples and should not be construed to narrow the scope or spirit of the disclosure in any way. It will be appreciated that the scope of the disclosure encompasses many potential embodiments, some of which will be further described below, in addition to those here summarized.

### BRIEF DESCRIPTION OF THE SEVERAL VIEWS OF THE DRAWINGS

Having thus described embodiments of the invention in general terms, reference will now be made to the accompanying drawings, which are not necessarily drawn to scale, and wherein:

FIG. 1 is an example geospatial arrangement, according to prior art;

FIG. 2 is a block diagram of a system used in the creation of logical units, according to some example embodiments;

FIG. 3 is a block diagram of an apparatus used in the creation of logical units, according to some example embodiments;

FIG. 4 is a flowchart of operations for creating a logical unit according to some example embodiments;

FIG. 5 is an example display used for creating a logical unit according to some example embodiments;

FIG. 6 is an example display for displaying a graphical logical unit according to some example embodiments;

FIG. 7 is an example display used for configuring a graphical logical unit according to some example embodiments; and

FIG. 8 is a flowchart of operations for creating a new logical unit based on an existing logical unit according to some example embodiments.

### DETAILED DESCRIPTION

Some embodiments of the present invention will now be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

As used herein, where a computing device is described to receive data from another computing device, it will be appreciated that the data may be received directly from the other computing device and/or may be received indirectly via one or more intermediary computing devices, such as, for example, one or more servers, relays, routers, network access points, and/or the like. Similarly, where a computing device is described herein to transmit data to another computing device, it will be appreciated that the data may be sent directly to the other computing device or may be sent to the other computing device via one or more interlinking computing devices, such as, for example, one or more servers, relays, routers, network access points, and/or the like.

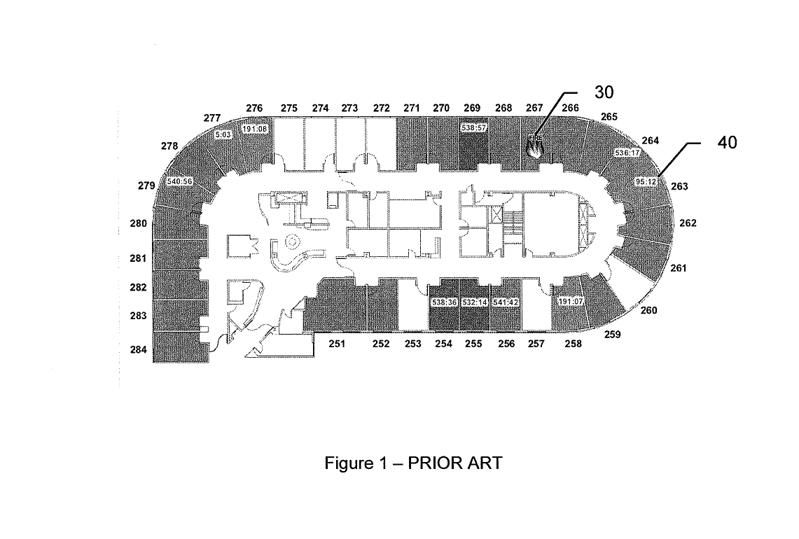

FIG. 1 illustrates a geospatial arrangement of a hospital floor. The geospatial arrangement provides information for the entire floor by room. Some rooms are color coded to provide statuses of rooms or patients in them. For example, for a cleaning crew, the geospatial arrangement may show color coded rooms to indicate which rooms are cleaned and which rooms are in need of cleaning. As an alternative use, the rooms may be color coded to indicate which rooms are occupied or vacant. Further, icon 30 indicates that an alarm has signaled in room 267, and timer 40 on room 263 indicates a countdown until a specified event or an elapsed time since a previously occurring event. The example geospatial arrangement may therefore provide pertinent information related to an entire floor of the hospital in a visual format easily interpreted by a user.

In practical use, a user may not need information for an entire floor, but may be more interested in particular rooms throughout a building and on different floors. According to an example embodiment, a user may create a logical unit, such that identified rooms may be displayed as a logical unit without regard to their respective geospatial arrangements. Although a hospital is used throughout as an example embodiment, it will be appreciated that the embodiments provided herein may be beneficial to many other fields and uses. The hospital environment is cited as a non-limiting example, and should not be construed to narrow the scope or spirit of the disclosure in any way.

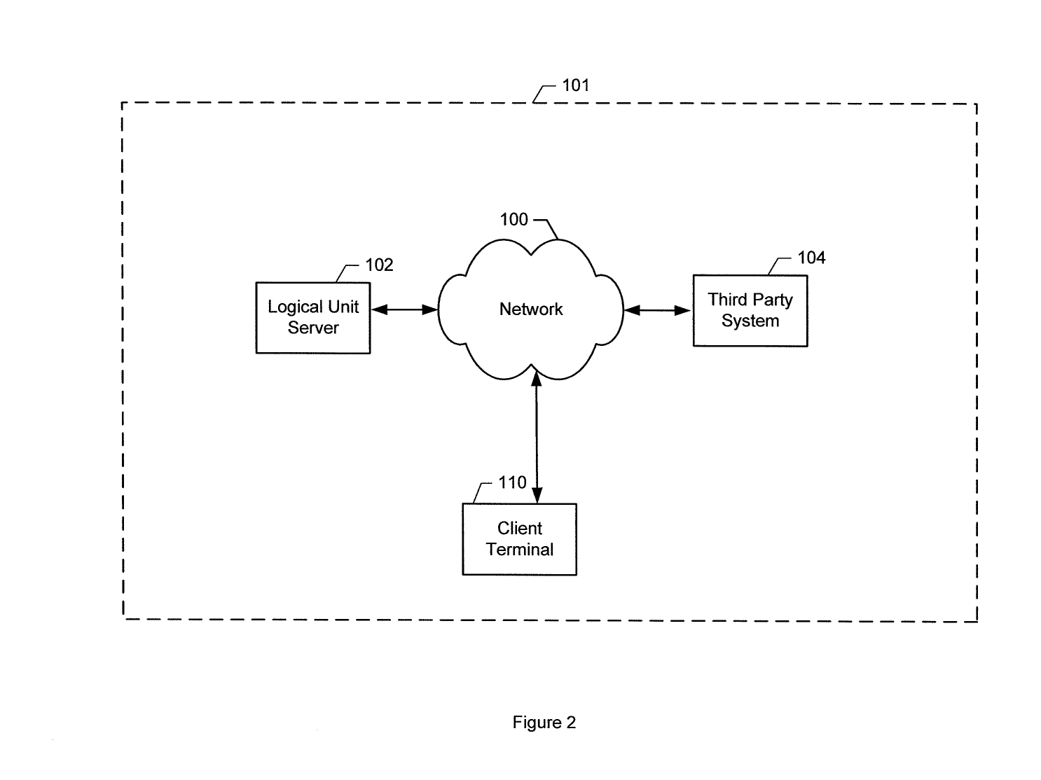

FIG. 2 illustrates a system 101 for creating logical units according to some example embodiments. It will be appreciated that the system 101, as well as the illustrations in other figures, are each provided as an example of an embodiment(s) and should not be construed to narrow the scope or spirit of the disclosure in any way. In this regard, the scope of the disclosure encompasses many potential embodiments in addition to those illustrated and described herein. As such, while FIG. 1 illustrates one example of a configuration of a system, numerous other configurations may also be used to implement embodiments of the present invention.

The system 101 may include a network 100, logical unit server 102, third party system 104, and/or a client, such as a client terminal 110. In some example embodiments, logical unit server 102 may be embodied as or comprise one or more computing devices. In some example embodiments, logical unit server 102 may be implemented as a distributed system or a cloud based entity that may be implemented within network 100. In this regard, logical unit server 102 may comprise one or more servers, a server cluster, one or more network nodes, a cloud computing infrastructure, some combination thereof, or the like.

Logical unit server 102 may be configured to maintain logical units and provide the graphical logical units for display by a client terminal 110. A logical unit may be considered data or information relative to a grouping of subunits, each subunit being defined based on a physical location (e.g., a room on a floor of a building, address, or other geographical location). A graphical logical unit may therefore refer to the display of such subunits, without regard to their respective geospatial arrangements.

According to the example embodiments provided herein, logical unit server 102 may be further configured to receive real-time or near real-time information related to any of the subunits, such as from third party system 104, for example, apply the information to a logical unit, and provide the graphical logical unit to client terminal 110. Graphical logical units may be provided to client terminal 110 via a web application, for example. In this regard, logical unit server 102 may additionally or alternatively be implemented as a web server. In some embodiments, raw data regarding the logical units may be transmitted from the logical unit server 102 to the client terminal 110, and an application installed on the client terminal 110 may generate the graphical logical unit for display.

The third party system 104 may be configured to communicate with logical unit server 102 over network 100, for example. Third party system 104 may be embodied as or comprise one or more computing devices. In some example embodiments, third party system 104 may be implemented as a distributed system or a cloud based entity that may be implemented within network 100. In this regard, third party system 104 may comprise one or more servers, a server cluster, one or more network nodes, a cloud computing infrastructure, some combination thereof, or the like.

In example embodiments provided herein, the third party system 104 may be operative to perform one or more functions associated with the infrastructure of a medical facility (e.g., hospital). For example, a third party system 104 may be used to systematically manage any information such as patient statuses, physician shift schedules, and/or nurse assignments, among others. The term ‘third party’ may be used to emphasize that the system 104 may operate independently from logical unit server 102 and/or under different ownership than that of the logical unit server 102, but it will be appreciated that in some embodiments, the third party system 104 may indeed be operated, separately, but nonetheless by the same entity in control of the logical unit server 102. In some embodiments, the third party system 104 and logical unit server 102 may be implemented on the same device.

Although FIG. 2 depicts one third party system 104, in some embodiments, any number of third party systems 104 may be present. In example embodiments provided herein, third party system 104 may be configured to provide pertinent information relative to a subunit (e.g. room) to the logical unit server 102, so that information gathered by the third party system 104 may be represented in a visual format on a graphical logical unit. In some embodiments, the data provided by third party system 104 may be associated to an entity without regard to physical location, such as a patient or staff member, for example. In such an embodiment, the logical unit server 102 may use room assignment data or other information (which may be provided by a third party system 104), to associate the entity to a physical location or subunit, such that visual representation of the information may be applied to a graphical logical unit.

Client terminal 110 may be embodied as a user terminal such as a laptop computer, tablet computer, mobile phone, desktop computer, workstation, or other like computing device. A client terminal 110 may be remote from the logical unit server 102 and/or third party system 104, in which case the user terminal 110 may communicate with any of the respective apparatuses via network 100. Additionally or alternatively, a client terminal 110 may be implemented on a third party system 104. User terminal(s) 110 may be used to access an application provided by the logical unit server 102, such as a maintenance tool to configure logical units, or a web application for viewing a graphical logical unit. As such, in example embodiments, physicians, staff, and/or other individuals may use client terminal 110 to view graphical logical units and related pertinent information provided by logical unit server 102.

Communication between client terminal 110 and logical unit server 102 may occur via network 100, and the client terminal 110 may request and receive data from the logical unit server 102 in such a manner that the logical unit server 102 operates as a blackbox server. Processing of the information and logical units may be performed by the logical unit server 102, deeming the client terminal 110, in some embodiments, a thin client. Any number of client terminals 110 may be present in system 101.

Network 100 may be embodied in a local area network, the Internet, any other form of a network, or in any combination thereof, including proprietary private and semi-private networks and public networks. The network 100 may comprise a wired network, wireless network (e.g., a cellular network, wireless local area network, wireless wide area network, some combination thereof, or the like), or a combination thereof, and in some example embodiments comprises at least a portion of the Internet.

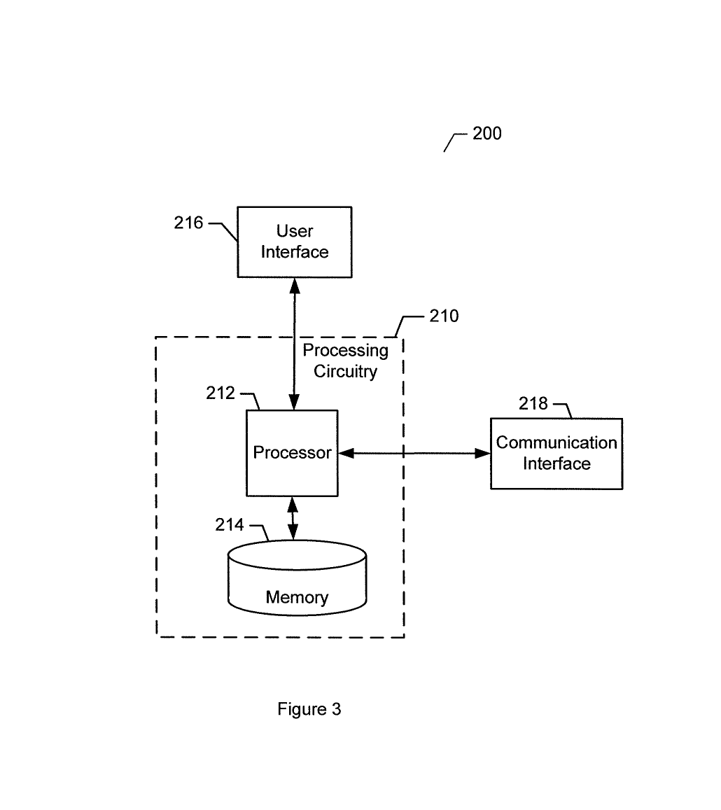

FIG. 3 illustrates an example apparatus 200 that may implement logical unit server 102, third party system 104, and/or client terminal 110, in accordance with some example embodiments. However, it should be noted that the components, devices, and elements illustrated in and described with respect to FIG. 3 below may not be mandatory and thus some may be omitted in certain embodiments. For example, FIG. 3 illustrates a user interface 216, as described in more detail below, which may be provided by the client terminal 110, but may be optional in the logical unit server 102 and/or third party system 104. Additionally, some embodiments may include further or different components, devices, or elements beyond those illustrated in and described with respect to FIG. 3.

Continuing with FIG. 3, processing circuitry 210 may be configured to perform actions in accordance with one or more example embodiments disclosed herein. In this regard, the processing circuitry 210 may be configured to perform and/or control performance of one or more functionalities of logical unit server 102, third party system 104, and/or client terminal 110 in accordance with various example embodiments. The processing circuitry 210 may be configured to perform data processing, application execution, and/or other processing and management services according to one or more example embodiments. In some embodiments, logical unit server 102, third party system 104, and/or client terminal 110, or a portion(s) or component(s) thereof, such as the processing circuitry 210, may be embodied as or comprise a computing device, e.g., an integrated circuit or other circuitry. The circuitry may constitute means for performing one or more operations for providing the functionalities described herein.

In some example embodiments, the processing circuitry 210 may include a processor 212, and in some embodiments, such as that illustrated in FIG. 3, may further include memory 214. The processing circuitry 210 may be in communication with or otherwise control a user interface 216, and/or a communication interface 218. As such, the processing circuitry 210 may be embodied as a circuit chip (e.g., an integrated circuit) configured (e.g., with hardware, software, or a combination of hardware and software) to perform operations described herein.

The processor 212 may be embodied in a number of different ways. For example, the processor 212 may be embodied as various processing means such as one or more of a microprocessor or other processing element, a coprocessor, a controller, or various other computing or processing devices including integrated circuits such as, for example, an ASIC (application specific integrated circuit), an FPGA (field programmable gate array), or the like. Although illustrated as a single processor, it will be appreciated that the processor 212 may comprise a plurality of processors. The plurality of processors may be in operative communication with each other and may be collectively configured to perform one or more functionalities of logical unit server 102, third party system 104, and/or client terminal 110 as described herein. The plurality of processors may be embodied on a single computing device or distributed across a plurality of computing devices collectively configured to function as logical unit server 102, third party system 104, and/or client terminal 110. In some example embodiments, the processor 212 may be configured to execute instructions stored in the memory 214 or otherwise accessible to the processor 212. As such, whether configured by hardware or by a combination of hardware and software, the processor 212 may represent an entity (e.g., physically embodied in circuitry—in the form of processing circuitry 210) capable of performing operations according to embodiments of the present invention while configured accordingly. Thus, for example, when the processor 212 is embodied as an ASIC, FPGA, or the like, the processor 212 may be specifically configured hardware for conducting the operations described herein. Alternatively, as another example, when the processor 212 is embodied as an executor of software instructions, the instructions may specifically configure the processor 212 to perform one or more operations described herein.

In some example embodiments, the memory 214 may include one or more non-transitory memory devices such as, for example, volatile and/or non-volatile memory that may be either fixed or removable. In this regard, the memory 214 may comprise a non-transitory computer-readable storage medium. It will be appreciated that while the memory 214 is illustrated as a single memory, the memory 214 may comprise a plurality of memories. The plurality of memories may be embodied on a single computing device or may be distributed across a plurality of computing devices collectively configured to function as logical unit server 102, third party system 104, and/or client terminal 110. The memory 214 may be configured to store information, data, applications, instructions and/or the like for enabling logical unit server 102, third party system 104, and/or client terminal 110 to carry out various functions in accordance with one or more example embodiments. For example, the memory 214 may be configured to buffer input data for processing by the processor 212. Additionally or alternatively, the memory 214 may be configured to store instructions for execution by the processor 212. As yet another alternative, the memory 214 may include one or more databases that may store a variety of files, contents, or data sets. For example, when apparatus 200 is implemented as logical unit server 102, the memory 214 may be configured to store logical units. Among the contents of the memory 214, applications may be stored for execution by the processor 212 to carry out the functionality associated with each respective application. In some cases, the memory 214 may be in communication with one or more of the processor 212, user interface 216, and/or communication interface 218, for passing information among components of logical unit server 102, third party system 104, and/or client terminal 110.

The user interface 216 may be in communication with the processing circuitry 210 to receive an indication of a user input at the user interface 216 and/or to provide an audible, visual, mechanical, or other output to the user. As such, the user interface 216 may include, for example, a keyboard, a mouse, a joystick, a display, a touch screen display, a microphone, a speaker, and/or other input/output mechanisms. As such, the user interface 216 may, in some example embodiments, provide means for user control of managing or processing data access operations and/or the like. In some example embodiments in which logical unit server 102 and/or third party system 104 is embodied as a server, cloud computing system, or the like, aspects of user interface 216 may be limited or the user interface 216 may not be present. Accordingly, regardless of implementation, the user interface 216 may provide input and output means in accordance with one or more example embodiments, such as displaying a graphical logical unit.

The communication interface 218 may include one or more interface mechanisms for enabling communication with other devices and/or networks. In some cases, the communication interface 218 may be any means such as a device or circuitry embodied in either hardware, or a combination of hardware and software that is configured to receive and/or transmit data from/to a network and/or any other device or module in communication with the processing circuitry 210. By way of example, the communication interface 218 may be configured to enable communication among logical unit server 102, third party system 104, and/or client terminal 110 via network 100. Accordingly, the communication interface 218 may, for example, include supporting hardware and/or software for enabling wireless and/or wireline communications via cable, digital subscriber line (DSL), universal serial bus (USB), Ethernet, or other methods.

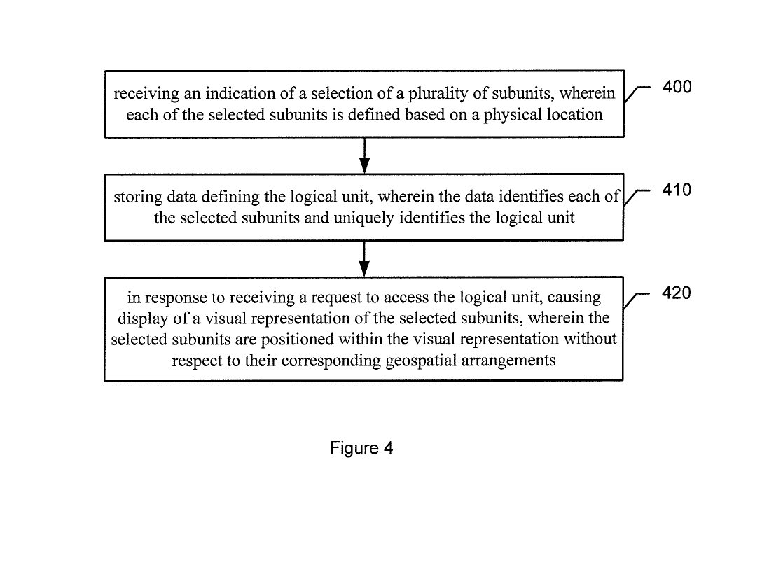

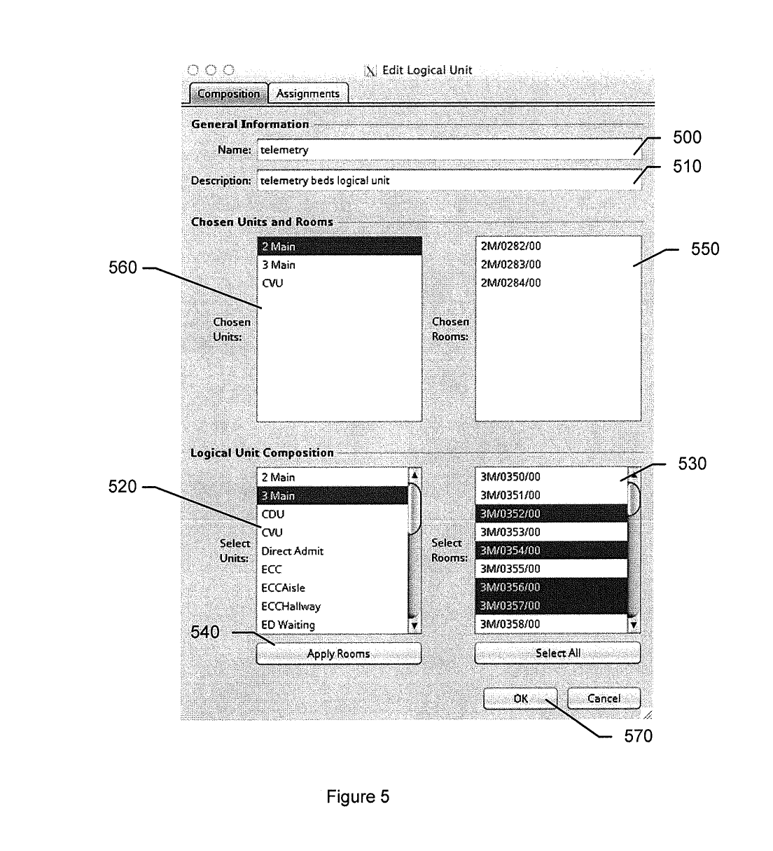

FIG. 4 is a flowchart illustrating example operations of logical unit server 102 according to an example embodiment. As shown by operation 400, the logical unit server 102 may be configured, such as with processor 212, memory 214, user interface 216, communication interface 218 and/or the like, for receiving an indication of a selection of a plurality of subunits, wherein each of the selected subunits is defined based on a physical location. A user may therefore access a user interface provided by the logical unit server 102 on client terminal 110, for example, to select any number of subunits, such as rooms in a building. An example display for selecting subunits is provided and described in more detail with respect to FIG. 5.

Although the subunits may be defined based on their physical location, such as room number and/or on a floor of a building, a user may desire to select rooms based on some other characteristic, such as a type of patient or treatment, practice group, patient status, meal selection, and/or level of care, for example. A user may therefore select some characteristic of a subunit other than the physical location, and the logical unit server 102 may identify the applicable subunits based on information provided by a third party system 104, for example.

As shown by operation 410, the logical unit server 102 may be configured, such as with processor 212, memory 214, and/or the like, for storing data defining the logical unit, wherein the data identifies each of the selected subunits and uniquely identifies the logical unit. A logical unit may therefore be named by a user to allow the user to later identify the logical unit. The name, or another unique identifier may be stored and associated with data identifying the subunits (e.g., any data identifying the rooms, such as room and floor numbers). The data may be stored to memory 214 so that it may be retrieved by a user requesting to view the graphical logical unit.

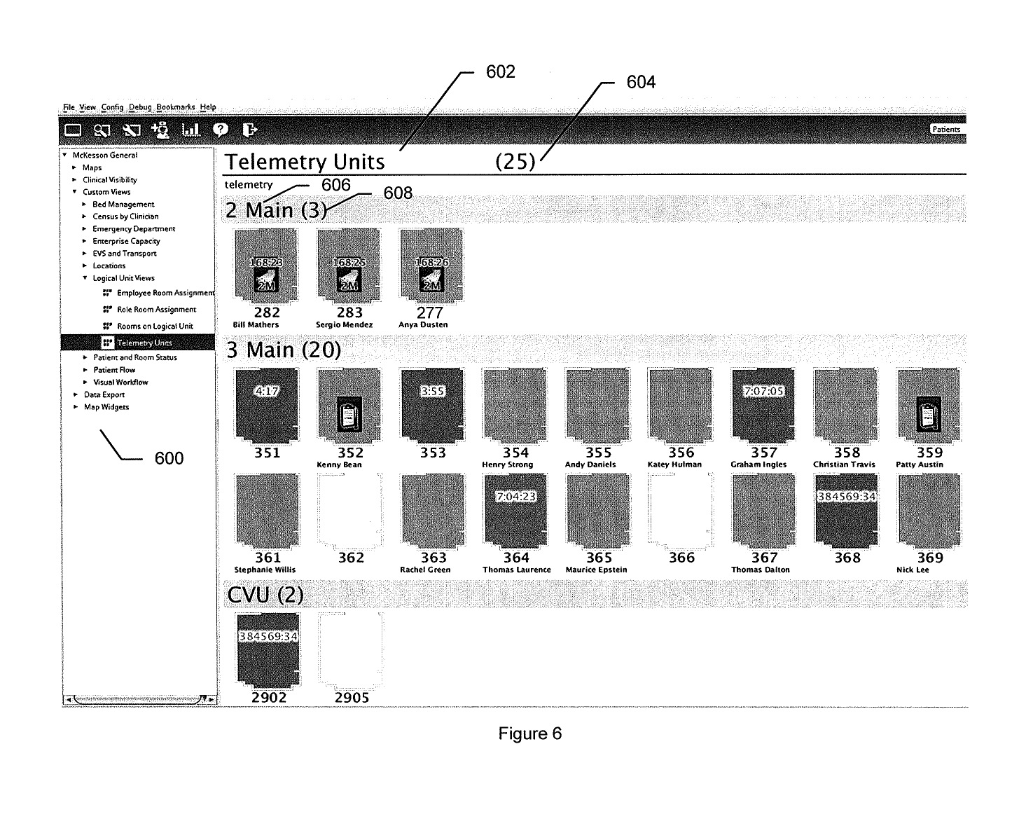

As shown by operation 420, the logical unit server 102 may be configured for, in response to receiving a request to access the logical unit, causing display of a visual representation (e.g., graphical logical unit) of the selected subunits, wherein the selected subunits are positioned within the visual representation without respect to their corresponding geospatial arrangements. An example of a visual representation, or graphical logical unit, is provided and described with respect to FIG. 6.

In some embodiments, the visual representation may convey pertinent information specific to at least one subunit. For example, information received from a third party system 104 may indicate that lunch is currently being served to a patient in a particular room. The logical unit server 102 may therefore apply an icon or other visual indication of the information to the particular room, and provide the graphical logical unit to the client terminal 110 for display.

In some embodiments, the visual representation may group the subunits based on their respective physical locations, such as a floor, for example. Regardless of whether the subunits are grouped, the subunits may be positioned or placed without respect to their physical locations relative to other subunits. Said differently, the subunits may be displayed or represented without respect to a geospatial arrangement, floor plan, or layout. While the example geospatial arrangement provided in FIG. 1 is a floor plan, it will be appreciated that a geospatial arrangement may be considered any data providing information regarding the physical location of a subunit with respect to another subunit, subunits, and/or all available subunits. Such embodiments may allow for improved utilization of a display screen to display the subunits, or rooms. Otherwise, a user may need to access several display screens to view different geospatial arrangements covering the subunits of interest. Using embodiments provided herein, a user may configure a graphical logical unit to provide information regarding rooms or subunits located on several different floors, on a single display screen, or reduced number of display screens, in comparison to viewing all applicable geospatial arrangements. The selected subunits of interest need not be physically adjacent. An example graphical logical unit is provided and described in further detail with respect to FIG. 6.

FIG. 5 is an example display used for creating or editing a logical unit. Such a display may be provided on a client terminal 110, for example. A user may provide a name and description of the logical unit in input areas 500 and 510, for example. A user may view available units in selection box 520. According to this example embodiment, a user may select a floor or other defined area in a building. In response, selection box 530 may update with available rooms (or subunits) in the selected unit. A user may then select the rooms of interest to be added to the logical unit. Upon selection of the “Apply Rooms” button 540, the selected rooms and respective units may be displayed in areas 550 and 560, respectively. Selection of a unit in area 560 may cause the area 550 to update to display only rooms from the selected unit to be included in the logical unit. In some embodiments, the rooms may not necessarily be categorized by floor or unit, but may be identified by room number alone. Selection of button 570 may result in the data being saved as a logical unit to the logical unit server 102. In embodiments in which the user is editing an existing logical unit, the appropriate data may be updated to reflect the changes. The logical unit may therefore be accessed and used to generate a graphical logical unit for subsequent requests.

FIG. 6 is an example display for displaying a graphical logical unit according to some example embodiments. The menu 600 appearing on the left side of the display may provide a hierarchy of available logical units, which may be grouped based on any categories such as locations, creator, purpose (e.g., capacity, bed management, etc.). From the menu 600, a user may select to view a particular logical unit, delete a logical unit, copy a logical unit as a new logical unit, and/or the like.

As shown by indicator 602, in this example, a user has selected to access a logical unit named “Telemetry Units.” The logical unit comprises 25 subunits, as indicated by indicator 604. The subunits identified by the logical unit are visually represented independently from their physical locations relative to one another and/or within a geographic area. In the example graphical logical unit, selected rooms are displayed and grouped by floor number, but without respect to a geospatial arrangement or a floor plan configuration such as provided in FIG. 1. Said differently, although the subunits are grouped by floor number, they are not positioned by geospatial arrangement or by their physical locations, such as according to a floor plan. The rooms are only merely grouped (e.g., placed under a subheading of their respective geographic area (e.g., floor number or identifier)), but not positioned relative to the geographic location of one another, such as according to their respective floor plans. On the other hand, subunits positioned relative to the geographic location of one another would resemble a floor plan or portion of floor plan such as that of FIG. 1. For example, in FIG. 6, rooms 277, 282, and 283 are displayed in a grouping of subunits on the floor “2 Main,” as shown by indicator 604, but other rooms on “2 Main” are not shown. Furthermore, the rooms 277, 282, and 283 are not positioned relative to the geographic location of one another. According to their respective geospatial arrangements, rooms 282 and 283 would be adjacent while a gap covering the space of 4 rooms would appear between 277 and 282. The graphical logical unit of FIG. 6 eliminates the gap and illustrates rooms 277, 282, and 283 without respect to their geospatial arrangements. Also note that in FIG. 1, room 277 is oriented differently when compared to rooms 282 and 283, but that in FIG. 6, the graphical logical unit presents the subunits in a relatively similar orientation. As such, in some embodiments, the subunits are displayed without respect to their orientations according to their respective geospatial arrangements.

Indicator 606 indicates the number of subunits grouped on the floor or in the specified geographic area described by indicator 606.

As such, in some embodiments, the selected subunits may be a subset of available subunits in which the subset includes less than all of the available subunits. The available subunits may include all rooms on a floor or all rooms in a building, for example. Based on the example graphical logical unit of FIG. 6, only a subset of all available rooms on “2 Main,” “3 Main” and “CVU” are selected as subunits. Further, in some embodiments, at least two of the selected subset of subunits may be physically located remotely from one another; (e.g., rooms 282 and 277 are considered to be located remotely from one another). In other words, the two rooms are not adjacent. In such embodiments, the visual representation may include only the selected subset of subunits, as opposed to all available subunits.

A user may therefore access a consolidated view of pertinent information regarding the selected rooms of interest. In this example, the subunits or rooms are color coded to illustrate room statuses. The assigned staff member for some rooms is also denoted below the visual representation of the subunit (e.g., room). Some particular rooms have associated icons indicating specific alerts or timers. Any of the third party systems 104 may provide the information to the logical unit server 102, so that the information may be visually applied to the graphical logical unit and to the appropriate subunit. The graphical logical unit therefore provides pertinent information to a user in a consolidated view, providing an efficient method for managing rooms located in different areas of a building.

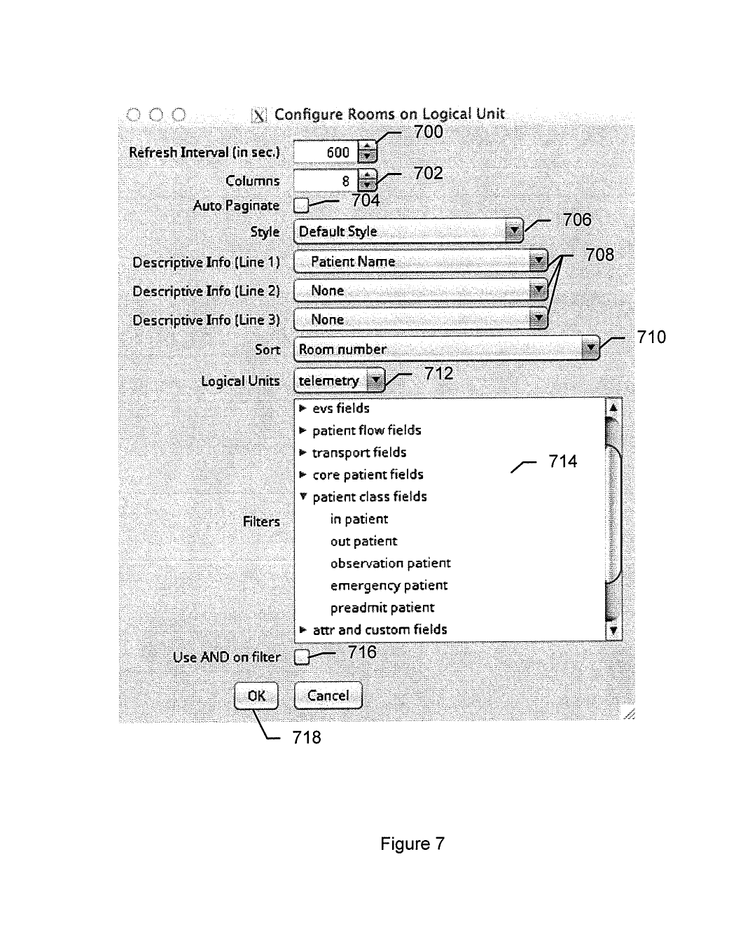

FIG. 7 is an example display used for configuring a graphical logical unit according to some example embodiments. In this regard, a user may access a display such as that of FIG. 7 to customize the graphical logical unit for viewing particular information and/or configurations. As shown by indicator 700, a user may indicate a refresh interval, in seconds, in which the graphical logical unit is updated. The refresh interval may therefore specify how often the information depicted on the graphical logical unit is updated. The logical unit server 102 may receive information from third party systems 104 on an ongoing basis, for example. Updates may be made to specific graphical logical units based on the refresh interval. A user working in a fast pace emergency environment may therefore configure refreshes to occur on a relatively short time interval, whereas a user monitoring statuses of stable patients, or the progress of meal service, for example, may configure refreshes to occur less frequently.

As shown by indicator 702, a user may configure the number of columns of subunits to be displayed, and/or auto paginate with indicator 704. Styles with which the graphical logical unit is displayed may be selected, such as with dropdown menu 706. Descriptive information to be displayed on the graphical logical unit, such as patient name, may be selected, such as in dropdown menus 708. The subunits may be sorted according to various factors, such as room number, such as with dropdown menu 710. Dropdown menu 712 allows for selecting specific logical units based on a predefined category. Selection box 714 allows a user to filter the displayed subunits or rooms based on various conditions and/or categorizations. The default use of the selection box 714 may result in subunits meeting any of the filtering requirements being displayed, while selection of indicator 716 may filter the displayed units such that only logical units meeting all the criteria are displayed. Selection of button 718 may cause the graphical logical unit to be displayed, reflecting the configurations entered with the display of FIG. 7. The configuration tool may therefore allow a user to tailor a particular logical unit for the user's personal preferences or practical usage.

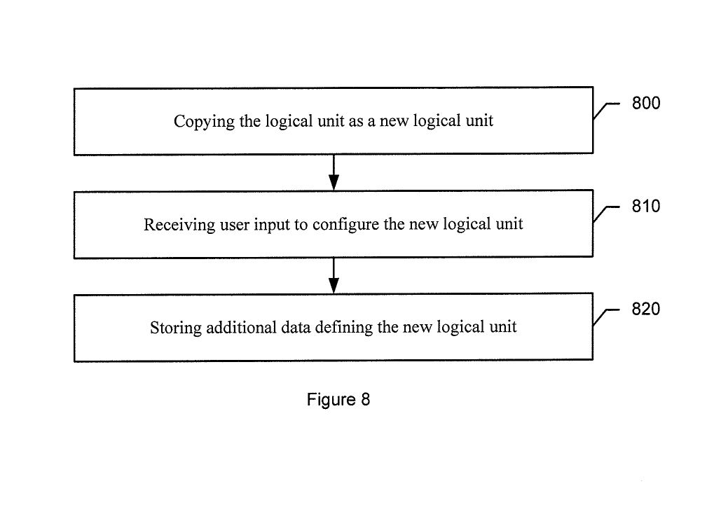

FIG. 8 is a flowchart of operations for configuring a logical unit according to some example embodiments. As shown by operation 800, the logical unit server 102 may be configured, such as with processor 212, memory 214, user interface 216, and/or the like, for copying the logical unit as a new logical unit. For example, a user, who may be the creator of an original logical unit, or user with access to the logical unit, may access a list of available logical units and request to clone (e.g., create an additional instance or copy of) the logical unit. As shown by operation 810, the logical unit server 102 may be configured, such as with user interface 216, communication interface 218, and/or the like, for receiving user input to configure the cloned, or newly created, logical unit. In this regard, a user may provide a name for the copied logical unit, and/or a description describing how the new logical unit differs from the original logical unit, for example. In some embodiments, the cloned version may be provided a new name by default so as to uniquely identify the cloned version from the logical unit from which it was cloned. A user may also edit the selected rooms, such as described with respect to operation 400 of FIG. 4. A user may edit a cloned version of a logical unit with a display such as the display of FIG. 5.

As shown by operation 820, the logical unit server 102 may be configured, such as with processor 212, memory 214, and/or the like, for storing additional data defining the new logical unit. The data defining the newly created logical unit may be stored as a new logical unit independently from the logical unit from which it was created. The data may be stored such as described with respect to operation 410 above.

As described above, embodiments described herein provide for creation and customization of graphical logical units. Logical units may be configured such that a user may view information regarding only the subunits or rooms the user is interested in monitoring. Third party system 104 may provide updates to pertinent information regarding each of the subunits so that a user may access the real time information on the user's customized logical unit. A user responsible for rooms located on several different floors of a hospital may therefore check the status of all the rooms using a single logical unit instead of repeatedly accessing and/or cycling through all applicable geospatial arrangements.

FIGS. 4 and 8 illustrate operations of a method, apparatus, and computer program product according to some example embodiments. It will be understood that each operation of the flowcharts or diagrams, and combinations of operations in the flowcharts or diagrams, may be implemented by various means, such as hardware and/or a computer program product comprising one or more computer-readable mediums having computer readable program instructions stored thereon. For example, one or more of the procedures described herein may be embodied by computer program instructions of a computer program product. In this regard, the computer program product(s) which embody the procedures described herein may comprise one or more memory devices of a computing device (for example, memory 214) storing instructions executable by a processor in the computing device (for example, by processor 212). In some example embodiments, the computer program instructions of the computer program product(s) which embody the procedures described above may be stored by memory devices of a plurality of computing devices. As will be appreciated, any such computer program product may be loaded onto a computer or other programmable apparatus (for example, logical unit server 102, third party system 104, and/or client terminal 110) to produce a machine, such that the computer program product including the instructions which execute on the computer or other programmable apparatus creates means for implementing the functions specified in the flowchart block(s). Further, the computer program product may comprise one or more computer-readable memories on which the computer program instructions may be stored such that the one or more computer-readable memories can direct a computer or other programmable apparatus to function in a particular manner, such that the computer program product may comprise an article of manufacture which implements the function specified in the flowchart block(s). The computer program instructions of one or more computer program products may also be loaded onto a computer or other programmable apparatus (for example, logical unit server 102, third party system 104, client terminal 110, and/or other apparatus) to cause a series of operations to be performed on the computer or other programmable apparatus to produce a computer-implemented process such that the instructions which execute on the computer or other programmable apparatus implement the functions specified in the flowchart block(s).

Accordingly, blocks of the flowcharts support combinations of means for performing the specified functions and combinations of operations for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

That which is claimed:

1. A method for creating a logical unit, the method comprising:
receiving an indication of a selection of a plurality of subunits, wherein each of the selected subunits is defined based on a physical location;
with processing circuitry, storing data defining the logical unit, wherein the data identifies each of the selected subunits and uniquely identifies the logical unit; and
in response to receiving a request to access the logical unit, causing display of a visual representation of the selected subunits, wherein the selected subunits are positioned within the visual representation without respect to their corresponding geospatial arrangements.

2. The method of claim 1, wherein receiving the indication of the selection of the plurality of subunits further comprises receiving an indication of a selection of a subset of all available subunits, wherein the subset comprises less than all of the available subunits, and wherein at least two of the selected subunits are physically located remotely from one another.

3. The method of claim 1, wherein the corresponding geospatial arrangements provide information regarding the subunits' physical location respective to other subunits.

4. The method of claim 1, wherein the visual representation conveys pertinent information specific to at least one subunit.

5. The method of claim 1, further comprising:
copying the logical unit as a new logical unit;
receiving user input to configure the new logical unit; and
storing additional data defining the new logical unit.

6. The method of claim 1, wherein each of the subunits comprises a room in a building, and the corresponding geospatial arrangements are defined by one or more floor plans.

7. The method of claim 1, wherein the selected subunits are grouped in the visual representation based on their respective physical locations.

8. The method of claim 1, wherein the plurality of subunits is selected based on a characteristic other than the respective physical locations.

9. A computer program product for creating a logical unit, the computer program product comprising at least one non-transitory computer-readable medium having computer-readable program instructions stored therein, the computer-readable program instructions comprising instructions, which when performed by an apparatus, are configured to cause the apparatus to at least:
receive an indication of a selection of a plurality of subunits, wherein each of the selected subunits is defined based on a physical location;
store data defining the logical unit, wherein the data identifies each of the selected subunits and uniquely identifies the logical unit; and
in response to receiving a request to access the logical unit, cause display of a visual representation of the selected subunits, wherein the selected subunits are positioned within the visual representation without respect to their corresponding geospatial arrangements.

10. The computer program product of claim 9, wherein receiving the indication of the selection of the plurality of subunits further comprises receiving an indication of a selection of a subset of all available subunits, wherein the subset comprises less than all of the available subunits, and wherein at least two of the selected subunits are physically located remotely from one another.

11. The computer program product of claim 9, wherein the corresponding geospatial arrangements provide information regarding the subunits' physical location respective to other subunits.

12. The computer program product of claim 9, wherein the visual representation conveys pertinent information specific to at least one subunit.

13. The computer program product of claim 9, wherein the computer-readable program instructions further comprises instructions, which when performed by an apparatus, are configured to cause the apparatus to at least:
copy the logical unit as a new logical unit;
receive user input to configure the new logical unit; and
store additional data defining the new logical unit.

14. The computer program product of claim 9, wherein each of the subunits comprises a room in a building, and the corresponding geospatial arrangements defined by one or more floor plans.

15. The computer program product of claim 9, wherein the selected subunits are grouped in the visual representation based on their respective physical locations.

16. The computer program product of claim 9, wherein the plurality of subunits is selected based on a characteristic other than the respective physical locations.

17. An apparatus for creating a logical unit, the apparatus comprising processing circuitry configured to communicate with a third party system and client, said processing circuitry configured to cause the apparatus to at least:
receive an indication of a selection of a plurality of subunits, wherein each of the selected subunits is defined based on a physical location;
store data defining the logical unit, wherein the data identifies each of the selected subunits and uniquely identifies the logical unit; and
in response to receiving a request to access the logical unit, cause display of a visual representation of the selected subunits, wherein the selected subunits are positioned within the visual representation without respect to their corresponding geospatial arrangements.

18. The apparatus of claim 17, wherein receiving the indication of the selection of the plurality of subunits further comprises receiving an indication of a selection of a subset of all available subunits, wherein the subset comprises less than all of the available subunits, and wherein at least two of the selected subunits are physically located remotely from one another.

19. The apparatus of claim 17, wherein the corresponding geospatial arrangements provide information regarding the subunits' physical location respective to other subunits.

20. The apparatus of claim 17, wherein the visual representation conveys pertinent information specific to at least one subunit.

21. The apparatus of claim 17, wherein the processing circuitry is further configured to cause the apparatus to at least:
copy the logical unit as a new logical unit;
receive user input to configure the new logical unit; and
store additional data defining the new logical unit.

22. The apparatus of claim 17, wherein each of the subunits comprises a room in a building, and the corresponding geospatial arrangements are defined by one or more floor plans.

23. The apparatus of claim 17, wherein the selected subunits are grouped in the visual representation based on their respective physical locations.

24. The apparatus of claim 17, wherein the plurality of subunits is selected based on a characteristic other than the respective physical locations.
